(19) United States
(12) Patent Application Publication
(10) Pub. No.: US 2023/0358273 A1
(43) Pub. Date: Nov. 9, 2023

(54) BEARING ARRANGEMENT AND ORTHOPAEDIC DEVICE HAVING SAME

(71) Applicant: OTTO BOCK HEALTHCARE PRODUCTS GMBH, Wien (AT)

(72) Inventors: Thomas ECKER-WESSELY, Eichgraben (AT); Christian BREUER-RUESCH, Wien (AT)

(21) Appl. No.: 18/246,224

(22) PCT Filed: Sep. 16, 2021
(86) PCT No.: PCT/EP2021/075473

§ 371 Date: Mar. 22, 2023

(30) Foreign Application Priority Data
Sep. 25, 2020 (DE) 10 2020 125 087.8

(51) Int. Cl. F16C 25/04

(52) U.S. Cl. CPC F16C 25/045; F16C 2316/10

(57) ABSTRACT

The invention relates to a bearing arrangement having a radial spherical plain bearing with an inner ring (10), which is arranged on a bolt (15) or pin and has a spherical outer contour (12), and with an outer ring (20), which has a hollow spherical inner geometry (22) for receiving the inner ring (10) and an outer geometry (21) for mounting in a bearing seat (30), wherein the inner ring (10) and the outer ring (20) are preloaded against one another in an axial direction via at least one preloading device (41, 42).

The invention relates to a bearing arrangement having a radial spherical joint with an inner ring, which is arranged on a bolt or pin and has a spherical outer contour, and with an outer ring, which has a hollow-spherical inner geometry for receiving the inner ring and an outer geometry for being received in a bearing seat, and also to an orthopedic device having such a bearing arrangement.

Orthopedic devices such as prostheses and orthoses, and also exoskeletons as special cases of orthoses, often have components that are movable in relation to one another and that are mounted on one another in particular in an articulated manner. In order to influence or control the relative movement between the two components, actuators or resistance devices can be arranged between the components. In addition to electric motor drives, it is also the case in particular that hydraulic dampers or hydraulic drives or else pneumatic dampers are arranged between the two components. Linear hydraulic systems are used as actuators or resistance devices particularly in the case of prostheses and orthoses of the lower extremities. The problem with linear drives and in particular with linear hydraulic systems is their sensitivity to bending moments. In order to decouple bending moments, it is possible for these linear hydraulic systems to be mounted spherically via spherical joints.

A radial spherical joint is understood to mean a bearing having an inner ring with a hollow-spherical, outwardly curved outer diameter and an outer ring with an inwardly curved, hollow-spherical inner diameter corresponding to the outer diameter of the inner ring, where the inner ring is received in the suitably curved outer ring. Spherical joints compensate for oscillating movements, pivoting movements and rotational movements at relatively low speeds. They likewise compensate for compensating movements between a shaft or axle, which is mounted in the inner ring, and a housing or bearing seat, in which the outer ring is mounted.

DE 10 2010 049 257 B4 discloses a prosthetic foot with a base spring and a connecting device, arranged above the base spring, for fastening the prosthetic foot to a prosthesis. The connecting device is coupled to the base spring in the forefoot region thereof via a frontal support and is supported on the base spring at the heel region via a damping element. The damping element is designed as a hydraulic damper, for example, and has at least one joint that is movable on multiple axes.

U.S. Pat. No. 8,603,190 B2 discloses an orthotic or prosthetic knee joint with a distal section and a proximal section that are mounted pivotably on each other. Between the distal section and the proximal section, a connecting piece, for example a hydraulic damper, is mounted so as to be pivotable about all three rotational degrees of freedom.

A spherical joint with a spherical bearing body and with a bearing shell is known from WO 2011/147039 A1, the bearing shell at least partially enclosing the bearing body. The bearing shell is designed in one piece and has a slit which is radial in relation to the wall thickness of the bearing shell and into which an insertion element can be introduced in order to adjust the radial play between the bearing shell and the bearing body.

The prior art has also disclosed spherical joints with a bearing body as inner ring and with a multi-part bearing shell, the two bearing shell parts being adjustable relative to each other in the axial direction.

The axial direction is the direction which runs in the longitudinal extent of the axis of the shaft or the bolt within the recess of the inner ring, while the radial direction extends orthogonally to the axial direction.

The spherical joints from the prior art basically have play or become worn, such that, during use, the existing play increases or play sets in. Such play can be heard and felt and is annoying particularly in the case of orthopedic devices. In addition, increasing play results in increasing wear. A reduction in the radial bearing play in order to avoid play means that the bearing friction increases and the bearing clearance required for operation is no longer available. This can result in the desired decoupling of bending moments not being achieved. In addition, there is the risk that the main movement of the component mounted in the spherical joint can no longer be carried out as desired in the event of increased friction caused by bracing.

The object of the present invention is therefore to make available an improved bearing arrangement with a radial spherical joint, and an improved orthopedic device having such a bearing arrangement.

According to the invention, this object is achieved by a bearing arrangement having the features of the main claim and by an orthopedic device having the features of the alternative independent claim. Advantageous embodiments and refinements of the invention are set out in the dependent claims, the description and the figures.

In the bearing arrangement having a radial spherical joint with an inner ring, which is arranged on a bolt or pin and has a spherical outer contour, and with an outer ring, which has a hollow-spherical inner geometry for receiving the inner ring and an outer geometry for mounting in a bearing seat, provision is made that the inner ring and the outer ring are pretensioned against each other in an axial direction via at least one pretensioning device. The at least one pretensioning device effects an axial pretensioning of the spherical joint, such that the two spherical bearing surfaces are always pressed against each other during use, by one bearing ring being pressed or pulled in one axial direction, while the other bearing ring is held in place or is pressed or pulled in the other direction. As a result, there is always contact between the outwardly curved outer contour of the inner ring and the inwardly curved and correspondingly spherical inner geometry of the outer ring. By means of the constant contact of the two surfaces of the outer contour and inner geometry, there are no sudden loads, and therefore wear and the development of noise are reduced.

The at least one pretensioning device also makes it possible to absorb axially acting forces and to ensure that the inner ring bears permanently on the outer ring. In addition, constant pretensioning is applied by the one pretensioning device over a comparatively long distance, such that tolerance compensation can be achieved in the case of increasing wear or of incorrect adjustment of the bearing clearance. The pretensioning in the axial direction enables rattle-free operation of the bearing arrangement, without the bearing clearance having to be reduced to a disruptive or restrictive degree.

In one embodiment, a first pretensioning device exerts an axially acting force on the outer ring or the inner ring. With a single pretensioning device, which exerts an axially acting force either on the outer ring or the inner ring, the play in the axial direction and thus also in the radial direction between the inner ring and the outer ring is effectively eliminated. The pretensioning device can be mounted on the outer ring or the inner ring and can transmit tensile or compressive forces. The design of the pretensioning device as a compression spring, for example, permits a space-saving, simple and reliable construction.

In a further embodiment, a second pretensioning device can be arranged such that an axial force, directed counter to the force of the first pretensioning device, is exerted on the respective other bearing part, in order to brace the two bearing rings against each other. If two elastic elements are arranged on the bearing arrangement, the degree of safety against failure increases. In addition, greater forces can be applied using comparatively small pretensioning devices.

In one embodiment, the pretensioning device is designed as an elastic element, a hydraulic and/or pneumatic system and/or an actuator or has such a component. The respective components can also be combined with one another, depending on the installation situation and on how much space is available for the arrangement.

An elastic element is designed, for example, as an elastomer body, as a compression spring, as a tension spring, as a leaf spring, as a disk spring or as a combination of these. If the pretensioning device is designed as a hydraulic and/or pneumatic system, a pressure accumulator, for example, acts on the fluid used and applies a pretensioning force to one of the bearing rings against the other bearing ring. The force is applied, for example, via a pressurized ram that bears on or acts on one of the bearing rings. The pressure accumulator can be designed as a mechanical spring accumulator and/or as a pneumatic pressure accumulator. If an actuator is provided and used, it can be coupled to a sensor that measures the force applied. If the measured value falls below a limit value, the actuator can be activated via a regulator or controller in order to adapt the desired pretensioning force. If two pretensioning devices which act on a respective bearing ring in mutually opposite directions are provided, they can be constructed in the same way or can have different configurations.

The pretensioning device or the pretensioning devices can generate a constant pressure or tension on one side mechanically, hydraulically, magnetically and/or pneumatically and can compensate for tolerances or wear during operation, without maintenance outlay, by applying a constant or quasi-constant pressure. In particular, the generation of a pretensioning force via magnetic components enables a combination with other principles of action, for example via springs, elastomer elements or other pretensioning devices. The outer ring can be mounted in a displaceable manner relative to the bearing seat, while the bearing seat is fixed axially with respect to lateral securing elements. The pretensioning force can then also take place between the bearing seat and the outer ring, with an axially fixed inner ring.

The pretensioning device can also act or be arranged between the inner ring and one of the securing elements, such that the two bearing surfaces are braced against each other when the rest is fixed accordingly.

In one embodiment of the pretensioning device as an elastic element, the elastic element has or the elastic elements have a spring characteristic with a slight gradient; in particular, the spring characteristic can be linear and have no more than a 10% reduction in force over the intended displacement path of the spring or of the elastomer element. In principle, the aim is for the gradient of the spring characteristic to be as shallow as possible, wherein the installation tolerances and manufacturing tolerances have to be taken into account, such that, even with a force reduction of around 25% over the displacement path of the components coupled to one another via the joint bearing, it is still possible to speak of a shallow gradient of the spring characteristic. The elastic element or the elastic elements can be pretensioned such that a sufficient pretensioning force acts in the axial direction even at the end of the compensating path or of the yielding, and the bearing rings are reliably braced against each other.

In one embodiment, at least one pretensioning device or one elastic element is assigned an adjusting device via which the pretensioning can be adjusted. This makes it possible to carry out an individual adjustment or to mount the inner ring with the outer ring without pretensioning. It is likewise possible to retrofit the pretensioning device and to apply the desired axial force via the adjusting device.

In one embodiment, the pretensioning device is supported or the pretensioning devices are supported on the bearing seat. The bearing seat in which the outer ring is mounted offers itself as an abutment, particularly when the pretensioning device acts on the inner ring and the outer ring is mounted axially immovably in the bearing seat. Alternatively, the pretensioning device can also be attached to the outer ring and can press against the inner ring or exert a tensile force in the axial direction, in order thereby to press or pull the inner ring onto the outer ring. Alternatively, it is possible to support the pretensioning device on the component mounted in the inner ring, for example a bearing bolt of a hydraulic damper, and to use the component as an abutment for the pretensioning device. If the pretensioning device is attached to the outer ring and is supported against the inner ring, or vice versa, the radial spherical joint can be manufactured and sold as one unit or as one module.

The pretensioning device can bear directly on the inner ring and/or the outer ring or can be coupled to the respective other bearing ring via a coupling element which, for example, has a friction-reducing surface or forms a good friction pairing with the inner ring or outer ring, respectively. The coupling element can be designed, for example, as a roller, sliding surface, rounded sliding element or the like.

The inner ring can be designed as a separate component and have a bore in which the bolt or pin is inserted and fixed. It can be fixed by pressing on, pressing in, shrinking, by mechanical fastening means, or else by cohesive bonding. Alternatively, the inner ring is designed as an integral component together with the pin or bolt; for example, a respective pin can protrude laterally from the inner ring in the axial direction in order to form the joint bearing.

The invention also relates to an orthopedic device, in particular a joint device having an upper part and a lower part and a resistance device or an actuator arranged between them, with a bearing arrangement as has been described above. In particular, hydraulic components or pneumatic components with a linear actuator are advantageously equipped with such a bearing arrangement. The bearing arrangement can be arranged, for example, on a piston rod and/or on a housing of a resistance device or a linear actuator. The linear actuator can also be designed as an active drive with a motor.

An illustrative embodiment of the invention is explained in more detail below with reference to the attached figures, in which:

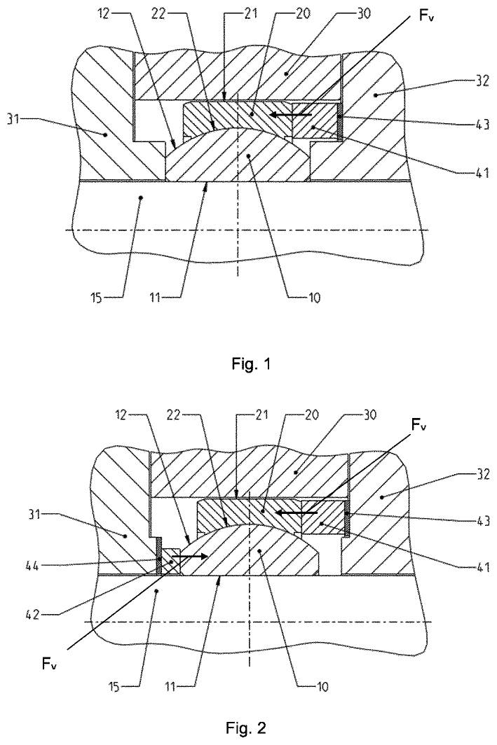

FIG. 1 shows a detailed view of a bearing arrangement in section;

FIG. 2 shows a variant of the bearing arrangement according to FIG. 1;

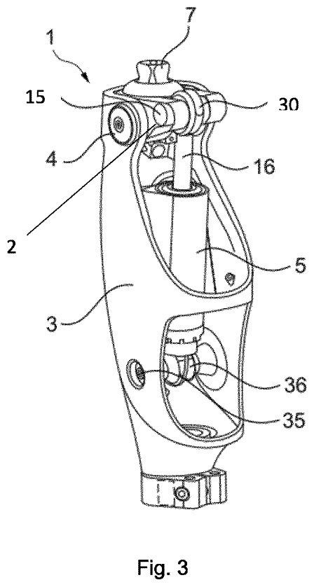

FIG. 3 shows a perspective view of a prosthetic joint device;

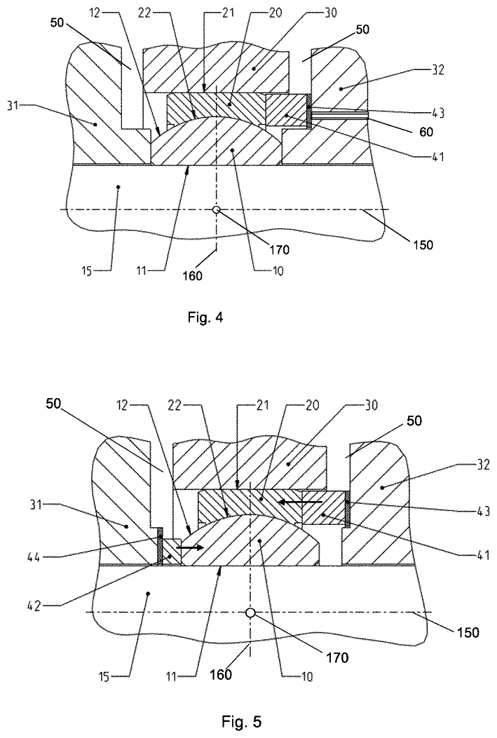

FIG. 4 shows a variant of FIG. 1;

FIG. 5 shows a variant of FIG. 2;

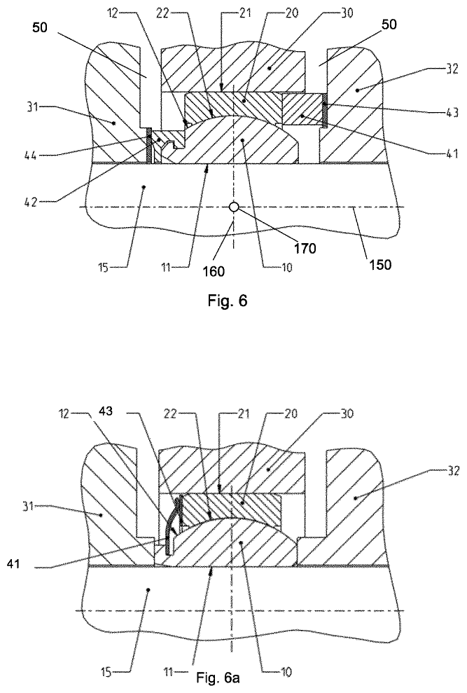

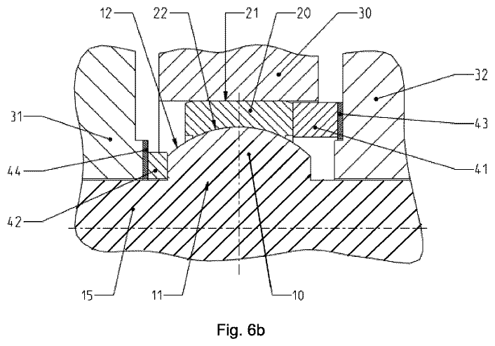

FIG. 6 shows a variant of FIG. 5;

FIG. 6a shows a variant of FIG. 6;

FIG. 6b shows a variant of FIGS. 6a, and

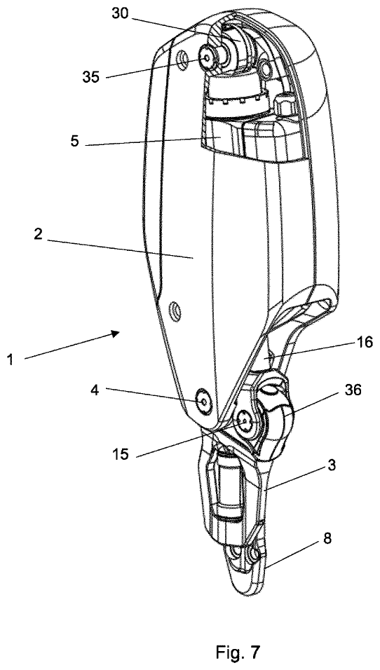

FIG. 7 shows a perspective view of an orthotic joint device.

FIG. 1 shows a partial sectional view of a bearing arrangement of a radial spherical joint with an inner ring 10 which is mounted on a bolt 15. The bolt 15, which is part of a hydraulic actuator for example, is inserted in a bore 11 of the inner ring 10 and secured against axial displacement along the longitudinal extent of the bolt 15 by securing elements 31, 32. The inner ring 10 is clamped between the two securing elements 31, 32 and clamped on the bolt 15 or on the axle accommodated within the bore 11. The securing elements 31, 32 can be screwed together or coupled to each other in some other way. The inner ring 10 also has an outer contour 12, which is spherical.

Furthermore, the bearing arrangement has an outer ring with a likewise spherical inner geometry 22. The inner geometry 22 is shaped corresponding to the outer contour 12 of the inner ring 10, with necessary bearing play being present between the outer ring 20 and the inner ring 10, so that the inner ring 10 can be moved relative to the outer ring 20. On account of the spherical configuration of both the outer contour 12 and the inner geometry 22, with the center points of the spherical inner geometry 22 and of the spherical outer contour 12 advantageously coinciding, it is possible for the bolt to be able to rotate relative to the outer ring 20 in three rotational degrees of freedom.

The outer ring 20 is mounted in a bearing seat 30 with a sliding fit there, such that the outer ring is movable in the axial direction. By contrast, the inner ring 10 is advantageously in an interference fit on the bolt 15 and cannot be moved axially on the bolt 15.

In the illustrative embodiment shown, an elastic element bears on the outer ring 20, as a pretensioning device 41 for providing a single axial pretensioning force in the direction of the outer ring 20, and is supported on a securing element 32 via a coupling element 43. The elastic element 41 is interposed between the outer ring and a component of the joint device other than the inner ring 10 or the outer ring 20. The pretensioning force does not act between two parts of a split outer ring which are braced against each other in order to adjust the bearing clearance, but is supported on a component outside the bearing rings. As an alternative to the arrangement of the coupling element 43 on the securing element 32, the coupling element 43 can also be arranged between the outer ring 20 and the elastic element 41. The coupling element 43 can facilitate a relative movement between the elastic element 41 and the securing element 32, for example by providing a favorable friction pairing. The coupling element 43 can also be used to fix the elastic element 41 to the securing element 32 or, in the case of a reverse arrangement, to the outer ring 20. Any play that may be present is caused by the pretensioned, elastic element 41 which exerts on the outer ring 20 a force acting in the axial direction. Play is thus compensated for by shifting of the outer ring 20 relative to the inner ring 10. The outer ring 20 is that part of the bearing arrangement which is or can be axially displaced by the elastic element 41 on account of the pretensioning force and thus compensates for play between the inner ring 10 and the outer ring 20.

FIG. 1 shows the pretensioning force Fᵥ acting in the axial direction, which force is directed to the left in the illustrative embodiment shown. As a result, the outer ring 20 is shifted relative to the inner ring 10, such that in FIG. 1 a right-hand subregion of the outer contour 12 is pressed against a correspondingly configured subregion of the inner geometry 22. On the left-hand side, the bearing gap thus increases, which cannot be seen on account of the small dimension of the bearing play. The pretensioning force Fᵥ ensures that a radial play is eliminated solely by compensating for the bearing play in the axial direction, without the bearing clearance between the inner ring 10 and the outer ring having to be reduced or changed. The application of an axial force is easily possible in design terms and does not require any complex components for expanding or reducing the circumference of the outer ring 20.

The sliding fit present between the outer ring 20 and the bearing seat 30 in the illustrative embodiment shown is merely one of several possibilities for eliminating radial play by compensating for axial play. An alternative set-up is one in which an enlarged gap is formed between at least one of the securing elements 31, 32 and the bearing seat 30, and the outer ring 20 is fixed axially in the bearing seat 30. The elastic element 41 can then act directly on the outer ring 20 or the bearing seat 30.

Alternatively, an elastic element can also be attached to the outer ring 20 or the inner ring 10 and be supported on the other ring, thus generating an axial force and a pretensioning between the two bearing rings.

FIG. 2 shows a structural set-up comparable to the bearing arrangement in FIG. 1. In addition to the first pretensioning device 41 in the form of a first elastic element, a second pretensioning device 42 is provided which exerts a compressive force Fᵥ on the inner ring 10, this pretensioning force likewise acting as a compressive force in the axial direction, but directed counter to the pretensioning force of the first elastic element 41. This has the effect that the right-hand side, as shown in the figure, of the inner ring 10 bears on the right-hand inner contour of the outer ring 20. The second elastic element 42 is also supported on a securing element 31, such that both forces are supported on components outside of the bearing rings 10, 20 on an abutment.

In principle, there is also the possibility that the pretensioning device 41, 42 is arranged on the outer ring and is supported on the inner ring 10 in order to bring about bracing and pretensioning in the axial direction. The introduction of the pretensioning force thus takes place on a component that is different from the inner ring or outer ring, respectively. When the axial pretensioning force is applied, the inner ring only ever bears on a surface region of the outer ring that is offset in the axial direction from the middle of the outer ring 20.

FIG. 3 shows a perspective view of an orthopedic joint device 1 in the form of a prosthetic knee joint. The orthopedic joint device 1 has an upper part 2 and a lower part 3 which are mounted on each other so as to be pivotable about a joint axis 4. The lower part 3 is designed as a three-dimensional hollow body which has an actuator or a damping device 5 with a piston rod 16. At its proximal end, the upper part 2 has a device 7 for fastening a proximal component, for example a thigh tube or a thigh socket. In the illustrative embodiment shown, the fastening device 7 is designed as a pyramid adapter; other designs are likewise possible. Moreover, a head as a bearing point or bearing seat 30, which is arranged on or attached to a proximal end of the piston rod 16, is mounted on the upper part 2 so as to be pivotable about an axle or pin 15, which will be explained in more detail later. Furthermore, the distal end of the damping device is mounted pivotably about an axis 35 at a distal bearing point or a distal bearing seat 36. The fixing of the damping device 5 both at the distal bearing point 36 and on the head or the proximal bearing seat 30 can be releasable, in particular via a screw connection or snap-fit connection.

In particular, the distal bearing point allows the damping device 5 to pivot exclusively about the pivot axis 35, which is oriented substantially parallel to the joint axis 4. Alternatively, a spherical joint with outer ring 20 and inner ring 10, as described with reference to FIG. 1 or 2, is arranged in the distal bearing seat 36. The head on the piston rod 16 serves in particular as a bearing seat 30 for a spherical joint with outer ring 20 and inner ring 10. The inner ring 10 is mounted on the bolt 15 and, on account of the spherical outer contour and the bearing in the corresponding spherical receptacle in the outer ring 20, the piston rod 16 performs a pivoting movement about the longitudinal extent of the bolt 15 and about the other two rotational degrees of freedom. As a result, the piston rod 16 is decoupled from bending moments when the upper part 2 pivots about the pivot axis 4. The same applies when the distal bearing seat 36 is mounted in a radial spherical joint.

The damping device 5, as shown in FIG. 3, can be in the form of a hydraulic damping device; a pneumatic damping device is also provided as an alternative. Damping devices which work on the basis of magnetorheological effects can also be used. As an alternative to a passive configuration, the damping device 5 can also be designed as an actuator, for example as an electric drive, which enables and permits a displacement of the upper part 2 relative to the lower part 3 through a corresponding connection and opposes this displacement with a resistance.

In addition to an embodiment of the orthopedic joint device 1 as a prosthetic knee joint, it can also be designed as an orthotic knee joint or another joint device.

FIG. 4 shows a variant of the embodiment according to FIG. 1 in a sectional view. Between the securing elements 31, 32 and the bearing seat 30, gaps 50 are arranged or formed on both sides, said gaps being larger compared to the embodiment according to FIG. 1. The bearing seat 30 is supported on the outer ring 20 and can move about a pivot axis 170 which is oriented perpendicularly to the plane of the drawing and runs through the center point of the radius of the outer contour 12 or of the inner geometry 22. In addition, by means of the gaps 50, the bearing seat 30 can pivot about a second pivot axis 160, which runs perpendicular to the longitudinal extent and longitudinal axis 150 of the bolt and lies in the plane of the drawing. Along with the pivotability about the longitudinal axis 150, movements of the bearing seat 30 relative to the bolt 15 or the securing elements 31, 32 about three rotational degrees of freedom or pivoting about the three axes 150, 160, 170 are thus possible. As in FIG. 1, the pretensioning element 41 acts in the axial direction along the longitudinal axis 150.

An adjusting device 60 in the form of an adjusting screw, which acts on the coupling element 43 and via which the pretensioning force of the pretensioning device 41 can be changed, is shown schematically in the right-hand securing element 32. If the adjusting screw 60 is screwed in in the direction of the outer ring 20, the pretensioning increases, and if it is turned in the opposite direction, the pretensioning decreases. The adjusting device 60 can be operated in a controlled manner via at least one sensor and can have an actuator (not shown). If magnetic components are used, the adjusting device 60 can also be designed as a device for changing the magnetic field strength.

FIG. 5 shows a variant of FIG. 2, in which there are also larger gaps 50 between the securing elements 31, 32 and the bearing seat 30. The two pretensioning elements 41, 42 are supported on opposing securing elements 31, 32 via the respective coupling elements 43, 44 and press the outer ring 20 or the inner ring 10 in opposite directions. As a result, the two bearing rings 10, 20 are braced against each other in the axial direction along the longitudinal axis 150 of the bolt 15. On account of the elasticity of the pretensioning elements 41, 42, rotation about all three pivot axes 150, 160, 170 is also possible.

FIG. 6 shows a variant of the embodiment according to FIG. 5. The basic set-up with the gaps 50 between the securing elements 31, 32 and the bearing seat 30 is retained. However, the cross-sectional shape of the pretensioning element 42 differs from the embodiment according to FIG. 5. A groove is worked on the one side in the inner ring 10, into which groove an annular projection of the pretensioning element 42 engages. The cross section of the pretensioning element 42 has an approximate inverted U-shape with a shortened right-hand limb which protrudes into the groove of the inner ring 10. The end of the other limb bears on the bolt 15. At the base, which is located at the top in the depiction in FIG. 6 and thus forms the outer circumference of the annular pretensioning element 42, a lateral thickening is formed which bears on the coupling element 44, such that a corresponding pretensioning force can build up away from the coupling element 44 in the direction of the other pretensioning element 41.

A variant of the embodiment according to FIG. 6 is shown in FIG. 6a. The basic set-up with the gaps 50 between the securing elements 31, 32 and the bearing seat is retained. However, the cross-sectional shape and the arrangement of the pretensioning element 42 differ from the embodiment according to FIG. 6. A groove is worked on the left-hand side in the inner ring 10, into which groove there engage(s) an annular projection or a plurality of radially inwardly pointing projections or tabs of the pretensioning element 41. The pretensioning element 41 is supported in the groove and bears, with a portion located outside of the groove or with a plurality of portions, on the coupling element 43. The pretensioning element 41 presses or pulls against the outer ring 20 and generates an axial pretensioning between inner ring 10 and outer ring 20. A groove can also be provided on both sides of the inner ring 10, such that one side is pressed and the other side is pulled. The advantage of this variant is that the system is designed in one assembly. The pretensioning element 41 can also be fixed on the outer ring 20 in a groove or another fastening device, for example hooked in, clipped in or held in a form-fitting or clamping manner with fastening elements such as clamps, clips, wedges or the like. It can also be fixed by force-fit engagement via magnets. The radially inner region of the pretensioning element 41 is supported, for example, in a groove in the inner ring or on a projection on the inner ring 10, such that a corresponding compressive or tensile force is applied in the axial direction. Here too, it is possible to use a plurality of pretensioning elements, which are arranged on both sides of the outer ring 20 in order to apply corresponding axial forces.

FIG. 6b shows a variant of FIG. 6a, in which variant, instead of a separate inner ring 10 which is placed on an axle or a bolt 15 and shrunk on, soldered on or welded on or in some other way fastened, the bolt 15 is designed in one piece with the inner ring 10. Such a one-piece design can also be used and formed in all the other embodiments of the bearing arrangement according to the figures and variants described above.

FIG. 7 shows, in a perspective view, an alternative use of both the bearing arrangement and the orthopedic device 1, namely as an orthosis component. An upper part 2 in the form of a housing for accommodating a linear actuator is arranged pivotably about a joint axis 4 on a lower part 3, which can be fastened to an orthosis rail, for example via a plate 8 articulated thereto. The upper part 2 is also fixed to an orthosis rail. The two orthosis rails (not shown) are then attached to a limb. The orthopedic device 1 thus forms a joint between the two orthosis rails, with the linear actuator 5 being able to be designed as a passive damping device or as an active drive with a motor. The linear actuator 5 has a piston rod 16 on which the bolt 15 is mounted. The inner ring is mounted on the bolt 15 and is supported on the bearing seat 30 via an outer ring 20. Conversely, the upper end of the linear actuator 5 is supported on the upper part 2 via a proximal bearing seat 36.

The pretensioning element or the pretensioning elements can also be designed as magnetic elements. Magnetic forces that repel or attract each other can be built up between the respective pretensioning element and the associated bearing ring. The pretensioning elements or the bearing rings can be designed as permanent magnets or can have permanent magnets. The respective other component can have oppositely oriented magnetic components or ferromagnetic elements. There is also the possibility of using a combination of elastic elements and magnetic elements, either as oppositely acting pretensioning devices or as mutually complementary and supporting elements of a pretensioning device. Magnetic components make it possible to transmit forces without mechanical wear, which means that maintenance is no longer necessary or that the maintenance intervals can be extended. If electromagnetic components are used, the respective axial pretensioning force can be set and adjusted through appropriate excitation. Wear can be compensated, for example, by increasing the excitation voltage or the current flow in the excitation coil.

1. A bearing arrangement having a radial spherical joint comprising:
a bolt or pin;
an inner ring arranged on the bolt or pin, wherein the inner ring has a spherical outer contour;
an outer ring, which has a hollow-spherical inner geometry for receiving the inner ring, and an outer geometry for mounting in a bearing seat; and
at least one pretensioning device,
wherein the inner ring and the outer ring are pretensioned against each other in an axial direction via the at least one pretensioning device.

2. The bearing arrangement as claimed in claim 1, wherein the at least one pretensioning device comprises a first pretensioning device which exerts a first axial force on the outer ring or the inner ring.

3. The bearing arrangement as claimed in claim 2, wherein the at least one pretensioning device comprises a second pretensioning device which exerts a second axial force directed counter to the first axial force of the first pretensioning device, and wherein the second axial force is exerted on either the inner ring or the outer ring whichever is not acted upon by the first axial force.

4. The bearing arrangement as claimed in claim 1 wherein the at least one pretensioning device is designed as an elastic element, a magnetic element, a hydraulic system, a pneumatic system, and/or has an actuator.

5. The bearing arrangement as claimed in claim 4, wherein the at least one pretensioning device is designed with the elastic element, and wherein the elastic element has a spring characteristic with a slight gradient.

6. The bearing arrangement as claimed in claim 1 further comprising an adjusting device via which the at least one pretensioning device is adjustable.

7. The bearing arrangement as claimed in claim 1 wherein the at least one pretensioning device is supported on the bearing seat, the inner ring, the outer ring, or a component mounted in a bore of the inner ring.

8. The bearing arrangement as claimed in claim 1 wherein the at least one pretensioning device bears directly on the inner ring and/or the outer ring or is coupled to the inner ring and/or the outer ring via a coupling element.

9. The bearing arrangement as claimed in claim 1 wherein the inner ring has a bore for receiving the bolt or pin, or wherein the inner ring is formed in one piece with the bolt or pin.

10. An orthopedic device having a bearing arrangement as claimed in claim 1.

11. The orthopedic device as claimed in claim 10, wherein the bearing arrangement is arranged on a linear actuator.
